(19) United States
(12) Patent Application Publication
(10) Pub. No.: US 2005/0150491 A1
(43) Pub. Date: Jul. 14, 2005

(54) STEAM INHALER

(76) Inventor: Yu-Yu Chen, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/755,290

(22) Filed: Jan. 13, 2004

(51) Int. Cl. A61M 15/00; A61M 16/10

(52) U.S. Cl. 128/203.16

(57) ABSTRACT

A steam inhaler includes a water tank with a heating chamber on a lateral side, a heater mounted on an external side wall of the heating chamber to generate water vapor, a vapor chamber at the front side of the water tank with a vapor inlet connected to the heating chamber such that vapor generated in the heating chamber flows to the vapor chamber, a connecting arm to convey vapor from the heating chamber, and a nozzle that atomizes the vapor from the connecting arm. The nozzle includes a rotatable cylinder with a vapor outlet port. The steam inhaler further includes a pair of ion electrodes on an inner wall of the connecting arm and projecting into the internal passage. Furthermore, negative ions are generated by a negative ion generator is mounted adjacent to the nozzle.

### BACKGROUND OF THE INVENTION

1. Field of the Invention

The present invention relates to a steam generating device, and more particularly to a steam inhaler.

2. Description of the Prior Art

A steam inhaler is a device for generating vapor by heating a tank of water by means of a heater. Conventionally, during operation, generated vapor is ejected and causes a medicinal liquid contained in a suction tank sucked up and atomized for inhalation into a patient's mouth for medical treatment or skin caring purpose.

There are a variety of steam inhalers known in the prior arts. Examples include the atomizing apparatus in U.S. Pat. No. 4,318,397, the inhalator in U.S. Pat. No. 4,604,999 and the steam inhaler in U.S. Pat. No. 4,942,874. Those prior arts possess different structures.

In some steam inhalers, because the arrangement of components is not good, in operation, some of the processes including the vaporization of water, ejection of vapor and/or suction of medicinal liquid are not ideal, and therefore the steaming effect is not poor. Although some conventional steam inhalers have improved structures, the structures are very complicated. Hence the assembly of the steam inhaler is complex and maintenance is not easy.

Also, most manufacturers usually do not put much effort in designing the vapor channels that are critical for good ejection of vapor. When vapor is generated and conveyed through the defective vapor channels, it is obstructed to produce a smooth ejection. Moreover, as the vapor is conveyed to the nozzle and ejected therefrom, hot dew is easily formed in the vicinity of the nozzle because of the structure of the nozzle. Such a phenomenon results to poor vapor atomization.

### SUMMARY OF THE INVENTION

Because of the aforesaid defects of the conventional steam inhaler, thus a primary object of the present invention is to provide a steam inhaler comprising a water tank, a heating chamber, a heater and a vapor chamber. With the good arrangement of the components, water is vaporized and flows through vapor passages smoothly, and is ejected from the nozzle.

Another object of the present invention is to provide a steam inhaler that possesses a rotatable nozzle, such that a user can adjust the ejecting direction for completely receiving the vapor.

A further object of the present invention is to provide a steam inhaler with an improved nozzle. The nozzle possesses a dew proof annulus that can reduce the formation of hot dew.

To achieve the above and other objects, in accordance with the present invention, there is provided with a steam inhaler comprising a water tank, a heating chamber arranged at a lateral side of the water tank, a heater which is mounted at an external side wall of the heating chamber for heating up the water in the heating chamber to generate vapor, a vapor chamber arranged at the front side of the water tank with a vapor inlet in communication with the heating chamber such that vapor generated in the heating chamber flows to the vapor chamber, a connecting arm for conveying the vapor from the heating chamber, and a nozzle which atomizes the vapor transported via the connecting arm.

Preferably, the nozzle is an angle adjustable structure. The nozzle includes a rotatable cylinder with a vapor outlet port. Vapor passes through an internal passage and the vapor outlet port of the rotatable cylinder to a nozzle head.

More preferably, the present invention further includes a pair of ion electrodes correspondingly arranged at an inner wall of the connecting arm and projecting into an internal passage of the connecting arm. Also, a negative ion generator is provided at a negative ion outlet in the vicinity of the nozzle for generating negative ions. The negative ion generator is embedded in the negative ion outlet. Alternatively, the negative ion generator is detachable from the steam inhaler.

### BRIEF DESCRIPTION OF THE DRAWINGS

The present invention will be apparent to those skilled in the art by reading the following description of the best mode and a preferred embodiment of a device for carrying out the present invention, with reference to the attached drawings, in which:

FIG. 1 is a perspective view of a steam inhaler constructed in accordance with the present invention;

FIG. 2 is an exploded view showing the components of the steam inhaler;

FIG. 3 is a perspective side view showing a heating chamber and a heater of the steam inhaler;

FIG. 4 is a sectional view taken along line 4-4 of FIG. 1;

FIG. 5 is a top schematic view of the steam inhaler;

FIG. 6 is an exploded view of a first embodiment of the steam inhaler constructed in accordance with the present invention, in which the steam inhaler is provided with an external housing;

FIG. 7 is a front perspective view of the steam inhaler of FIG. 6 in assembly;

FIG. 8 is a top schematic view showing a cover, connecting arm and nozzle;

FIG. 9 is an exploded view showing the components of the nozzle;

FIG. 10 is a sectional view taken along line 10-10 of FIG. 8;

FIG. 11 is a sectional view taken along line 11-11 of FIG. 8;

FIG. 12 is an enlarged sectional view of a dew proof annulus of the steam inhaler;

FIG. 13 is an enlarged sectional view of a negative ion generator of the steam inhaler;

FIG. 14 is a partial sectional view showing that a draining control rod is inserted into a horizontal sleeve at the bottom of a water tank;

FIG. 15 is an exploded view of a second embodiment of the steam inhaler constructed in accordance with the present invention, in which the steam inhaler is provided with an external housing; and

FIG. 16 is a front perspective view of the steam inhaler of FIG. 15 in assembly.

### DETAILED DESCRIPTION OF THE PREFERRED EMBODIMENTS

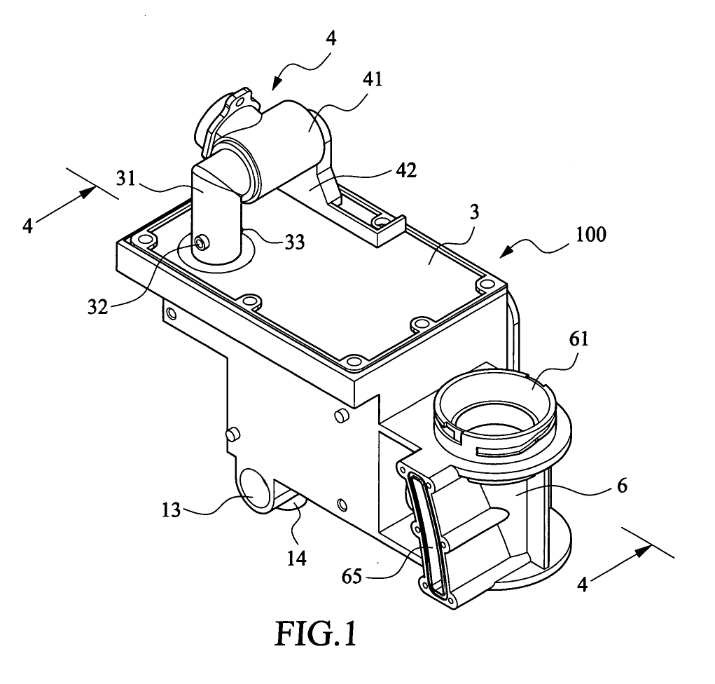

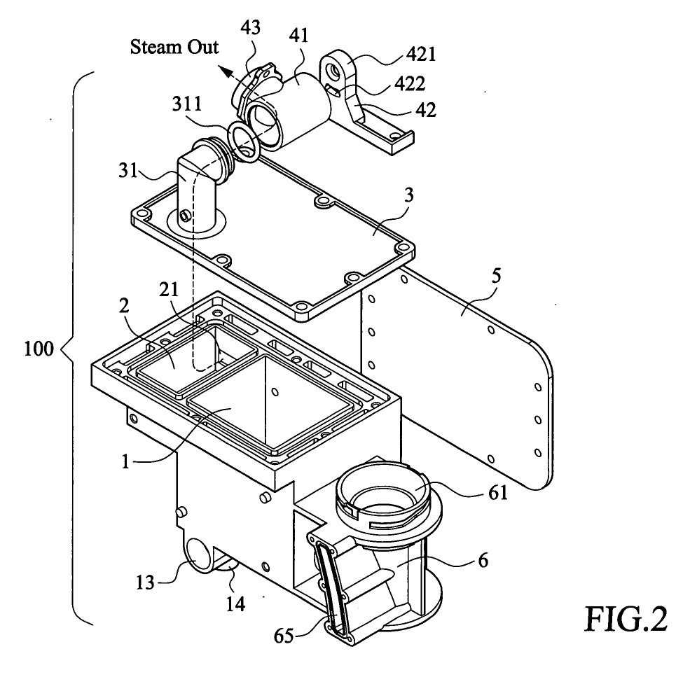

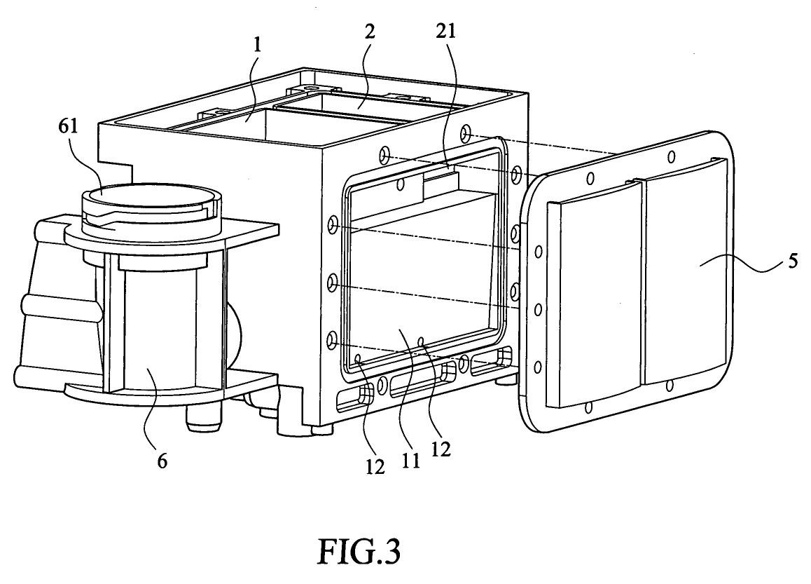

With reference to the drawings and in particular to FIGS. 1 to 3, FIG. 1 shows a perspective view of a steam inhaler of the present invention; FIG. 2 is an exploded view showing the components of the steam inhaler; FIG. 3 is a perspective side view of the steam inhaler. The steam inhaler 100 comprises a water tank 1 with an open top for storage of water.

Arranged at a front side of the water tank 2 is a vapor chamber 2 with an open top. A heating chamber 11 is arranged at a lateral side of the water tank 1 that is provided with at least one opening 12 at the lower part in communication with the heating chamber 11. A vapor inlet 21 is formed at the side wall of the vapor chamber 2 in communication with the heating chamber 11.

The tops of the water tank 1 and the vapor chamber 2 are covered by a cover 3 that is mounted with a connecting arm 31. The connecting arm 31 is in connection with a nozzle 4.

As shown in FIG. 3, a heater 5 is provided at an external side wall of the heating chamber 11 for heating up the water in the heating chamber 11. Water is boiled and vaporized. The vapor passes through the vapor inlet 21 to the vapor chamber 2, and then flows via the connecting arm 31 to the nozzle 4 for atomization.

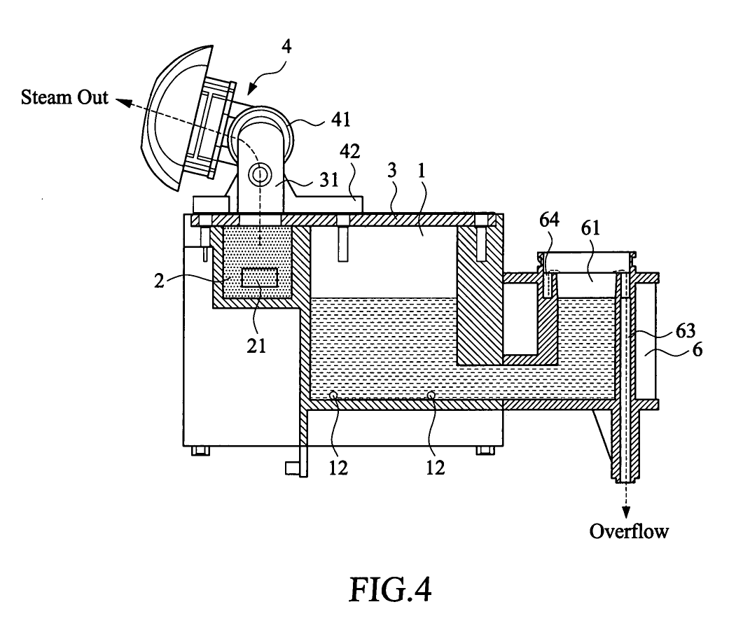

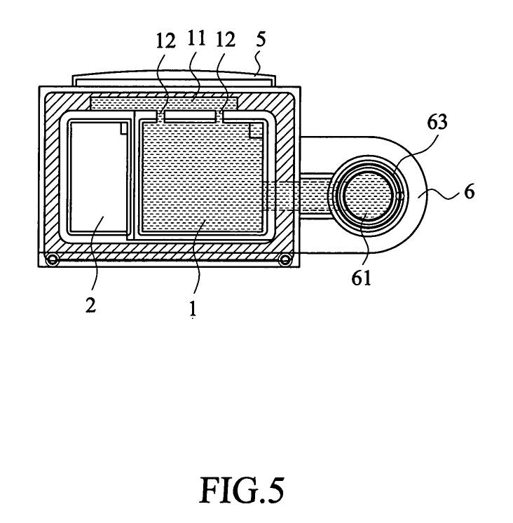

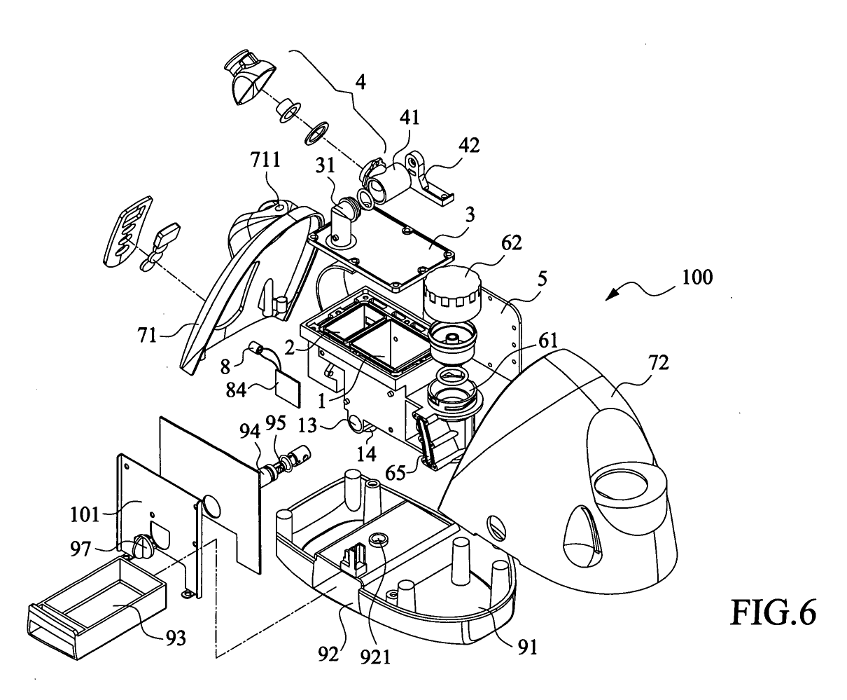

Please refer to FIGS. 4 to 6. FIG. 4 is a sectional view taken along line 4-4 of FIG. 1; FIG. 5 is a top schematic view of the steam inhaler; FIG. 6 is an exploded view of a first embodiment of the steam inhaler in accordance with the present invention, in which the steam inhaler is provided with an external housing.

A feeding tank 6 is mounted at a rear side of the water tank 1. A user can fill in water via a mouth 61 which is covered by a lip 62. The lip 62 may be formed with threads and screwed to the mouth 61. The lower part of the feeding tank 6 is communicated with the water tank 1. When the feeding tank 6 is filled with water, water flows to the water tank 1 freely. Accordingly, the water tank 1 and the feeding tank 61 have the same water level.

Please also refer to FIG. 4. As shown, the feeding tank 6 is formed with an overflow channel 63 and a leading trough 64 surrounding an upper edge of the feeding tank 6. When too much water is filled in exceeding the capacity of the feeding tank 6, excess water is guided to flow into the leading trough 64 and flows out through the overflow channel 63. Furthermore, a see-through water level gauge 65 is provided at a lateral side of the feeding tank 6 for observing the water level in the feeding tank 6, such that the user can check the amount of residual water in the feeding tank 1.

Also, the steam inhaler 100 is provided with an external housing designed in accordance with requirements. As shown in the first embodiment of the steam inhaler in FIG. 6, the housing may be comprised of a front casing 71 and a rear casing 72. The front casing 71 is formed with a negative ion outlet 711 in the vicinity of the nozzle 4. A negative ion generator 8 is embedded in the negative ion outlet 711.

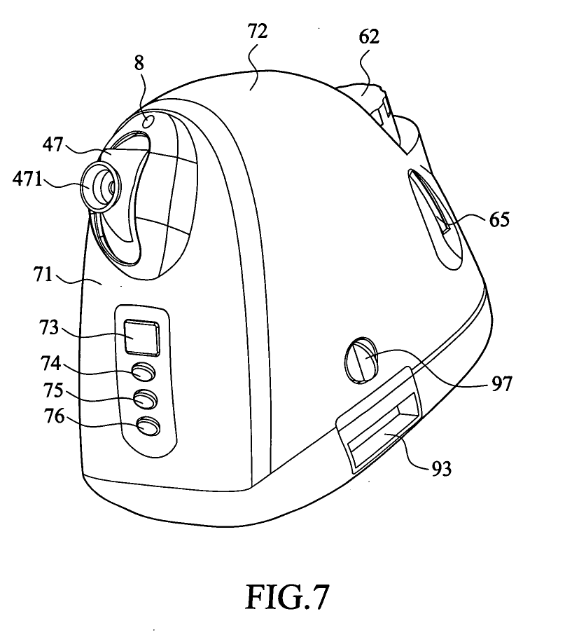

FIG. 7 is a perspective front view of the steam inhaler of FIG. 6. A display 73 is disposed at an appropriate position of the front casing 71 for displaying the operating status of the steam inhaler 100. A power switch 74 is provided for turning on/off of the steam inhaler 100. Also, an ion vapor button 75 is provided for starting or terminating the generation of ions, and a negative ion vapor button 76 is provided for starting or terminating the generation of negative ions.

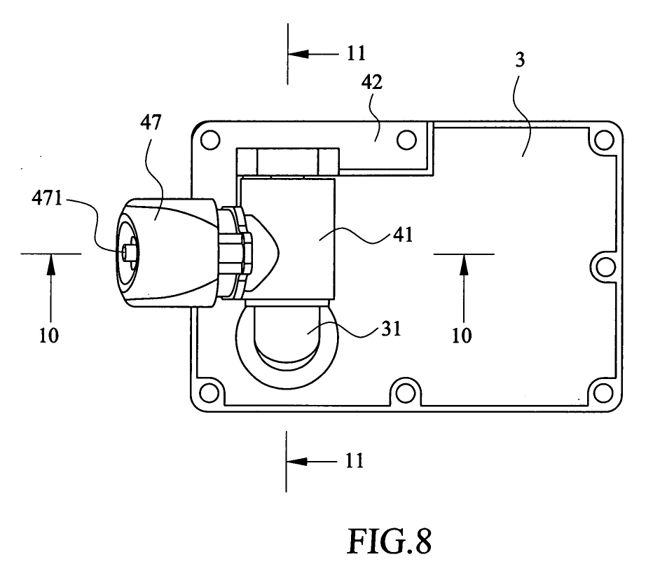

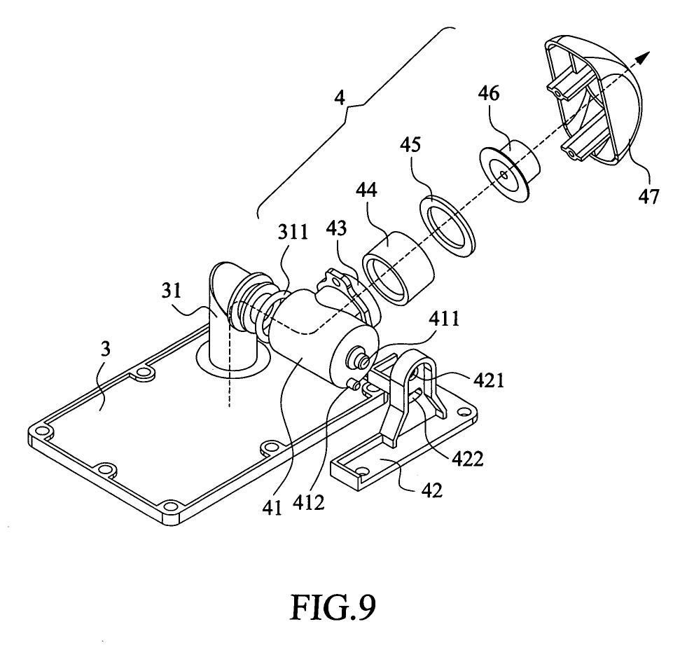

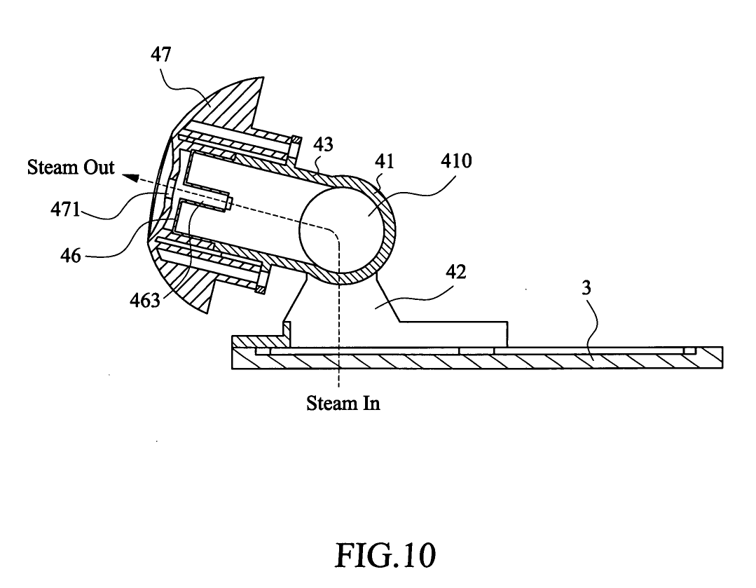

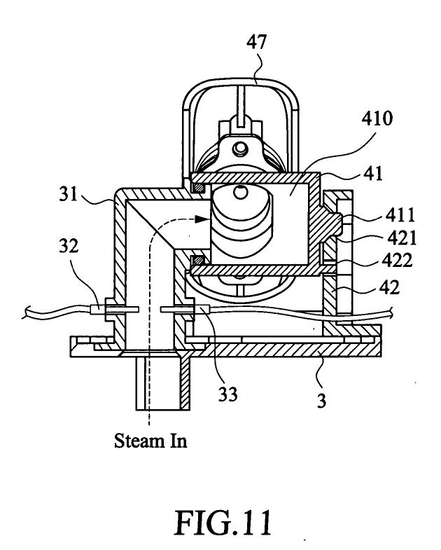

Please refer to FIGS. 8 to 11. FIG. 8 is a schematic top view showing a cover plate, a connecting arm and a nozzle; FIG. 9 is an exploded view showing the components of the nozzle; FIG. 10 is a sectional view taken along line 10-10 of FIG. 8; FIG. 11 is a sectional view taken along line 11-11 of FIG. 8.

The nozzle 4 comprises a rotatable cylinder 41 that is formed with an internal passage 410 therein. The rotatable cylinder 41 has an open end for connection to an upper end of the connecting arm 31 by a conventional way of engagement that allows free rotation. A sealing ring 311 is sandwiched between the connecting arm 31 and rotatable cylinder 41 to prevent leakage.

The other end of the rotatable cylinder 41 is closed and formed with a shaft 411 projecting out from the rotatable cylinder 411. A stand 42 is fastened on the cover 3 and formed with a bore 421 for insertion of the shaft 411. By means of the engagement between the bore 421 and the shaft 411, the rotatable cylinder 41 is supported and secured at a predetermined position of the steam inhaler 100 and is able to rotate around a central axis of the shaft 411.

Moreover, a locking pin 412 is formed at the close end of the rotatable cylinder 41 adjacent to the shaft 411. A restricting channel 422 is formed at the stand 422 corresponding to the locking pin 412, such that when the locking pin 42 passes through the restricting channel 422, it is restricted to move within the restricting channel 422. Thereby, when the user turns the rotatable cylinder 41, the rotatable cylinder 41 is restricted to rotate within an allowable range of angle.

At the middle section of the rotatable cylinder 41, a vapor outlet port 43 is formed. The vapor outlet port 43 is mounted in sequence with an annulus 44, a sealing ring 45, a dew proof annulus 46 and a nozzle head 47. By means of the arrangement, the vapor that flows to the rotatable cylinder 41 from the vapor chamber 2 via the connecting arm 31 is conveyed to the vapor outlet port 43 and finally atomized from a central opening 471 at a front end of the nozzle head 47.

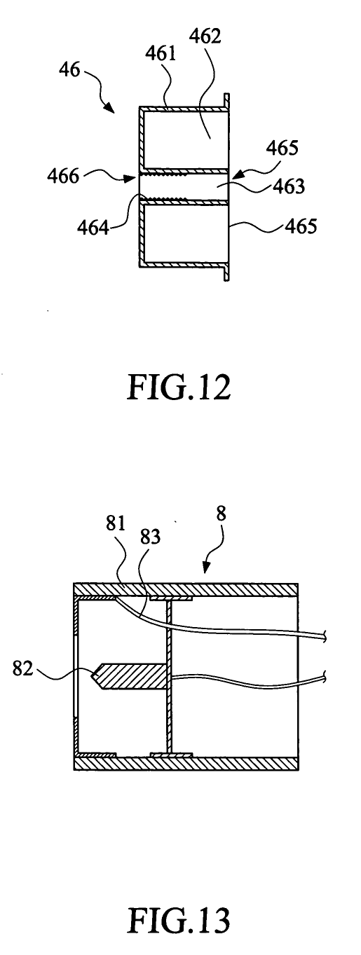

FIG. 12 is an enlarged sectional view of the dew proof annulus of the steam inhaler. The dew proof annulus 46 is made of aluminium or stainless steel and is formed with a concentric U-shaped section 461. The concentric U-shaped section 461 defines a space therein and has an open end and a close end. The dew proof annulus 46 is also formed with a central through hole 463 along the axial direction.

When vapor is conveyed to an inflow end 465 of the central through hole 463 of the dew proof annulus 46 and flows out from an outflow end 466, because of the structure of the dew proof annulus 46, the formation of hot dew is largely reduced.

Besides, a part of the central though hole 463 near the outflow end 466 is formed with a rough inner surface 464 in sawtooth form, preventing the condensed droplets to be carried away by the vapor flowing therethrough.

Also, a pair of ion electrodes 32, 33 are correspondingly arranged at an inner wall of the connecting arm 31. The ion electrodes 32, 33 project into an internal passage of the connecting arm 31 and are connected with electrical wires to a control circuit (not shown). As vapor flows through the connecting arm 31, it is ionized by the ion electrode 32, 33 and forms ions. Thereby, the steam inhaler can generate vapor containing ions.

FIG. 13 is an enlarged sectional view of the negative ion generator of the present invention. The negative ion generator 8 comprises an annular body 81 with an internal space. A first electrode 82 is disposed in the center of the annular body 81, and a second electrode 83 is positioned near to the first electrode 82. The first and second electrodes 82, 83 are connected respectively to a circuit board 84 by wires. The control circuit on the circuit board 84 controls the negative ion generator 8 to generate negative ions, and thereby the steam inhaler 100 is able to generate vapor with negative ions. Thus, the user can select pure vapor, vapor containing ions, or vapor containing negative ions in accordance with his requirements.

Please refer to FIG. 6. The steam inhaler 100 comprises a base 91 at the bottom. The base 91 is formed with a chamber 92 for accommodating a drip tray 93 putting in from a lateral opening. An aperture 921 is formed at the top of the chamber 92.

A longitudinal horizontal sleeve 13 is formed at the bottom of the water tank 1, which is communicated with a draining hole 14. The draining hole 14 is positioned in correspondence with the aperture 921 at the chamber 92.

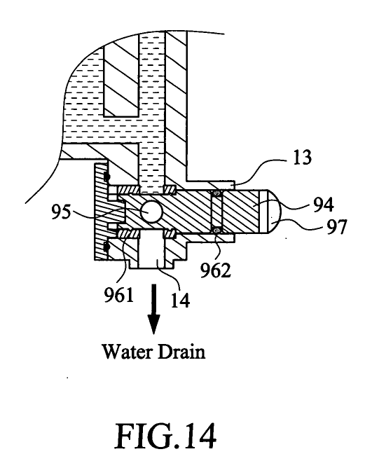

A draining control rod 94, which comprises a diametrical perforation 95, is inserted into the horizontal sleeve 13. When the draining control rod 94 is put in the sleeve 13 at a second position, the diametrical perforation 95 is aligned with the draining hole 14, forming a continuous passage. As shown in FIG. 14, to prevent water leakage, a conventional waterstop rubber 961 is mounted around the draining control rod 94 in the vicinity of the diametrical perforation 95, and a sealing ring 962 is mounted around the draining control rod 94 contacting the horizontal sleeve 13. The draining control rod 94 protrudes out of a side wall 101 of the steam inhaler 100 and is mounted with a rotatable drain button 97 to control the draining control rod 94 for drainage of water.

When the draining control rod 94 is at a first position, the perforation 95 is aligned perpendicularly to the draining hole 14. Therefore, water is not able to drain from the perforation 95. When the user turns the draining control rod 94 to the second position by means of the rotating drain button 97, the perforation 95 is aligned with the draining hole 14 and forms a continuous passage. Accordingly, water in water tank 1 drains through the perforation 95 and draining hole 14. Subsequently, water is conveyed through the aperture 921 to the drip tray 93 in the chamber 92.

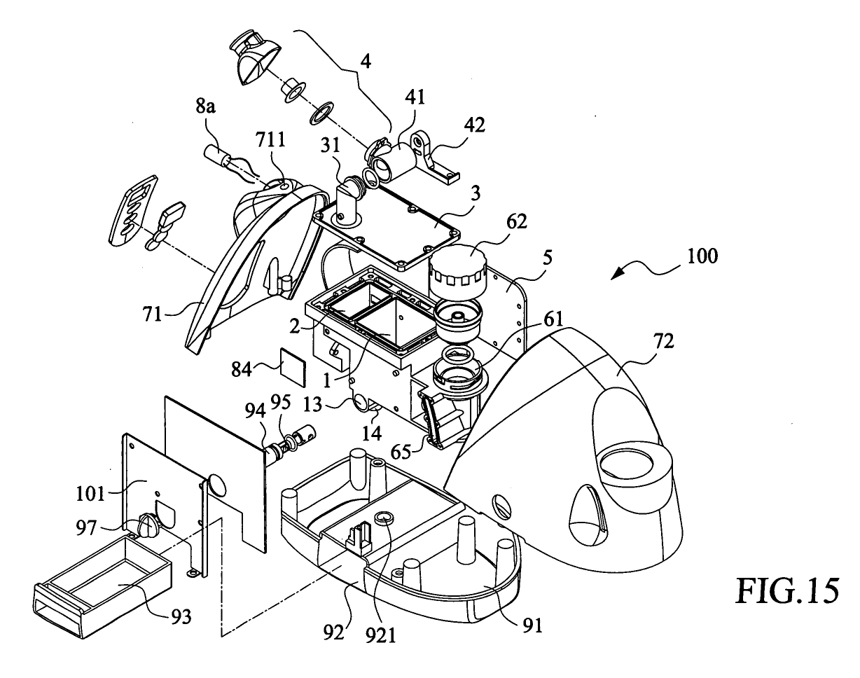

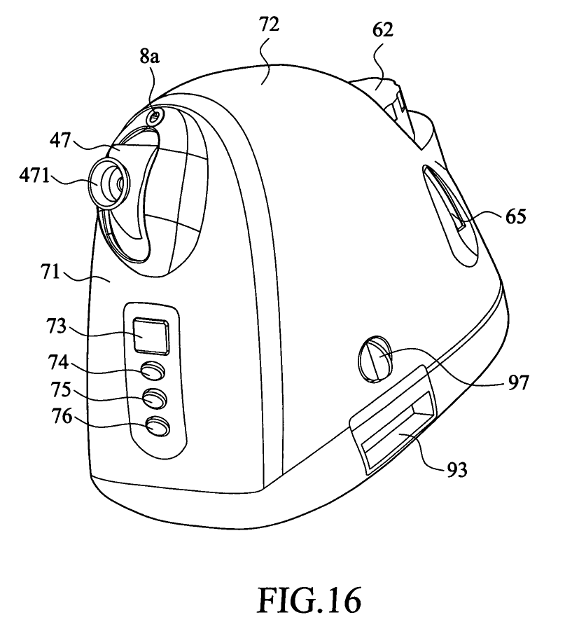

FIG. 15 is an exploded view of a second embodiment of the steam inhaler in accordance with the present invention and FIG. 16 is a front perspective view of the steam inhaler of FIG. 15 in assembly. In the embodiment, the steam inhaler 100 is mounted with a detachable negative ion generator 8a. The user can assemble the negative ion generator 8a with the steam inhaler 100 or remove the negative ion generator 8a from the steam inhaler 100 according to his requirements. The other components of the steam inhaler 100 are same as that in the first embodiment as shown in FIGS. 6 and 7.

Although the present invention has been described with reference to the preferred embodiments thereof, it is apparent to those skilled in the art that a variety of modifications and changes may be made without departing from the scope of the present invention which is intended to be defined by the appended claims.

1. A steam inhaler comprising:
a water tank for storage of water;
a heating chamber, which is arranged at a lateral side of the water tank and formed with at least one opening at a lower part of the heating chamber in communication with the water tank;
a heater, which is arranged at a side wall of the heating chamber for heating up the water in the heating chamber;
a vapor chamber, which is arranged at a front side of the water tank and formed with a vapor inlet at the side wall in communication with the heating chamber for transportation of vapor from the heating chamber;
a connecting arm, which is arranged at a top of the vapor chamber for conveying the vapor therein;
a nozzle, which is connected to an upper end of the connecting arm for atomizing the vapor flowing from the connecting arm; and,
a feeding tank arranged at a rear side of the water tank and comprising a mouth for filling with water, with a lower part of the feeding tank being in fluid communication with the water tank to permit water to flow freely from the feeding tank to the water tank,
wherein the feeding tank is formed with an overflow channel and a leading trough surrounding an upper edge of the feeding tank such that when too much water is filled in exceeding the capacity of the feeding tank, excess water is conveyed to the leading trough and flows out through the overflow channel.

2. (canceled)

3. (canceled)

4. The steam inhaler as claimed in claim 1, wherein the feeding tank is provided with a see-through water level gauge at a lateral side of the feeding tank.

5. The steam inhaler as claimed in claim 1, wherein the nozzle comprises:
a rotatable cylinder, which is formed with an internal passage therein and has an open end and a close end, the open end connecting rotatably to the upper end of the connecting arm with a sealing ring sandwiched therebetween;
a vapor outlet port, formed at a middle section of the rotatable cylinder and being in communication with the internal passage of the rotatable cylinder such that vapor from the connecting arm flows through the internal passage and flows out from the vapor outlet port;
a shaft, projecting out from the close end of the rotatable cylinder; and
a stand, which is formed with a bore for insertion of the shaft, and thereby secures the rotatable cylinder in a predetermined position and allows the rotatable cylinder to rotate around a central axis of the shaft.

6. The steam inhaler as claimed in claim 5, wherein the close end of the rotatable cylinder is formed with a locking pin adjacent to the shaft and a restricting channel is formed at a position of the stand corresponding to the locking pin for insertion of the locking pin, and when the user turns the rotatable cylinder, the rotatable cylinder is restricted to rotate within a range of angle allowed by the restricting channel.

7. The steam inhaler as claimed in claim 5, wherein the nozzle further comprises a dew proof annulus which is formed with an internal space with a open end and a close end and a central through hole along the axial direction, and which is connected to the vapor outlet port to prevent the vapor that is conveyed from an inflow end of the central through hole to an outflow end of the central through hole to form hot dew.

8. The steam inhaler as claimed in claim 7, wherein a part of the central through hole near the outflow end is formed with a rough inner surface in sawtooth form, preventing the condensed droplets to be carried away by the vapor flowing therethrough.

9. The steam inhaler as claimed in claim 1, further comprising:
a base, which is arranged at a bottom of the water tank and formed with a chamber;
a drip tray, accommodating in the chamber;
an aperture, formed at a top of the chamber corresponding to a draining hole beneath a horizontal sleeve at the bottom of the water tank; and
a draining control rod, which comprises a diametrical perforation and is inserted into the horizontal sleeve, and when the draining control rod is at a second position, the diametrical perforation is aligned with the draining hole, forming a continuous passage;
wherein when the draining control rod is at a first position, the diametrical perforation is aligned perpendicularly to the draining hole, and therefore water in the water tank is not able to drain from the perforation, and when the user turns the draining control rod to the second position, the perforation is aligned with the draining hole and forms a continuous passage, and accordingly, water in the water tank drains through the perforation, the draining hole and the aperture to the drip tray in the chamber.

10. The steam inhaler as claimed in claim 1, wherein the connecting arm is provided with a pair of ion electrodes which are correspondingly arranged at an inner wall of the connecting arm and project into the internal passage of the connecting arm.

11. The steam inhaler as claimed in claim 1, wherein a negative ion outlet is formed in the vicinity of the nozzle and a negative ion generator is embedded in the negative ion outlet for generating negative ions.

12. A steam inhaler comprising:
a water tank for storage of water;
a heater, which is arranged at a lateral side of the water tank for heating up the water in the water tank to generate vapor;
a vapor chamber, which is arranged at a front side of the water tank and formed with a vapor inlet in communication with the water tank, through which vapor generated in the water tank flows into the vapor chamber;
a connecting arm, which is arranged at a top of the vapor chamber for conveying the vapor therein; and
a rotatable nozzle, which is connected to an upper end of the connecting arm for atomizing the vapor from the connecting arm, the rotatable nozzle comprising:
a rotatable cylinder, which is formed with an internal passage therein and has an open end and a close end, the open end connecting rotatably to an upper end of the connecting arm with a sealing ring sandwiched therebetween; and
a vapor outlet port, formed at the middle section of the rotatable cylinder and being in communication with the internal passage of the rotatable cylinder such that vapor from the connecting arm flows through the internal passage and flows out from the vapor outlet port.

13. The steam inhaler as claimed in claim 12, wherein the nozzle further comprises a stand, which is formed with a bore for insertion of a shaft projecting out from the close end of the rotatable cylinder, and thereby the rotatable cylinder is secured in predetermined position and allowed to rotate around a central axis of the shaft.

14. The steam inhaler as claimed in claim 12, wherein the close end of the rotatable cylinder is formed with a locking pin adjacent to the shaft and a restricting channel is formed at a position of the stand corresponding to the locking pin for insertion of the locking pin, and when the user turns the rotatable cylinder, the rotatable cylinder is restricted to rotate within a range of angle allowed by the restricting channel.

15. The steam inhaler as claimed in claim 12, wherein a feeding tank is arranged at a rear side of the water tank, the feeding tank comprising a mouth for filling of water and a lower part of the feeding tank being in communication with the water tank, such that water flows freely from the feeding tank to the water tank.

16. The steam inhaler as claimed in claim 12, wherein the nozzle further comprises a dew proof annulus which is formed with an internal space with a open end and a close end and a central through hole along the axial direction, and which is connected to the vapor outlet port to prevent the vapor that is conveyed from an inflow end of the central through hole to an outflow end of the central through hole to form hot dew.

17. The steam inhaler as claimed in claim 12, wherein a part of the central through hole near the outflow end is formed with a rough inner surface in sawtooth form, preventing the condensed droplets to be carried away by the vapor flowing therethrough.

18. The steam inhaler as claimed in claim 12, further comprising:
a base, which is arranged at a bottom of the water tank and formed with a chamber;
a drip tray, accommodating in the chamber;
an aperture, formed at a top of the chamber corresponding to a draining hole beneath a horizontal sleeve at the bottom of the water tank; and
a draining control rod, which comprises a diametrical perforation and is inserted into the horizontal sleeve, and when the draining control rod is at a second position, the diametrical perforation is aligned with the draining hole, forming a continuous passage;
wherein when the draining control rod is at a first position, the diametrical perforation is aligned perpendicularly to the draining hole, and therefore water is not able to drain from the perforation, and when the user turns the draining control rod to the second position, the perforation is aligned with the draining hole and forms a continuous passage, and accordingly, water in water tank drains through the perforation, the draining hole and the aperture to the drip tray in the chamber.

19. The steam inhaler as claimed in claim 12, wherein the connecting arm is provided with a pair of ion electrodes which are correspondingly arranged at an inner wall of the connecting arm and project into the internal passage of the connecting arm.

20. The steam inhaler as claimed in claim 12, wherein a negative ion outlet is formed in the vicinity of the nozzle and a negative ion generator is embedded in the negative ion outlet for generating negative ions.
